(19) United States
(12) Patent Application Publication
(10) Pub. No.: US 2024/0002924 A1
(43) Pub. Date: Jan. 4, 2024

(54) PREPAKAGED POLYMERASE CHAIN REACTION (PCR) REAGENT FOR NUCLEIC ACID AMPLIFICATION AND METHOD THEREOF

(71) Applicant: Xiamen University, Xiamen (CN)

(72) Inventors: Chimeng TZENG, Xiamen (CN); Xueting WANG, Xiamen (CN)

(21) Appl. No.: 18/337,091

(22) Filed: Jun. 19, 2023

Related U.S. Application Data
(63) Continuation of application No. PCT/CN2021/138691, filed on Dec. 16, 2021.

(30) Foreign Application Priority Data
Dec. 17, 2020 (CN) 202011500628.X

(51) Int. Cl. C12Q 1/686

(52) U.S. Cl. CPC C12Q 1/686

(57) ABSTRACT

A prepackaged polymerase chain reaction (PCR) reagent for nucleic acid amplification and a method thereof are provided. A PCR reagent includes an antifreeze protein and an antifreeze protein antibody.

### RELATED APPLICATIONS

This application a continuation of International patent application PCT/CN2021/138691, filed on Dec. 16, 2021, which claims priority to Chinese patent application 202011500628.X, filed on Dec. 17, 2020. International patent application PCT/CN2021/138691 and Chinese patent application 202011500628.X are incorporated herein by reference.

### REFERENCE TO AN ELECTRONIC SEQUENCE LISTING

The contents of the electronic sequence listing (SequenceListing.xml; Size: 12,348 bytes; and Date of Creation: Jun. 19, 2023) is herein incorporated by reference.

### FIELD OF THE DISCLOSURE

The present disclosure relates to a prepackaged polymerase chain reaction (PCR) reagent for nucleic acid amplification and a method thereof.

### BACKGROUND OF THE DISCLOSURE

Polymerase chain reaction (PCR) has played an important role in biological experiments since being invented. In particular, quantitative real-time PCR (qPCR) techniques have become increasingly important in microbiological testing. The qPCR is a PCR technique that allows quantitative monitoring of a copy number of a target gene. In the qPCR, fluorescent dye or a fluorescent marker to a PCR system is added to produce fluorescence in combination with an amplification product. Changes in fluorescence signals of the PCR system are directly detected by a fluorescence signal detector, and an amount of PCR amplification and an amount of initial nucleic acid in the target gene of an sample is quantitatively estimate by plotting of fluorescence intensity versus a number of PCR cycles. This technique has advantages of high sensitivity, short detection time, and low cross-contamination.

Components required for the PCR comprise DNA polymerase, a primer, a probe, dNTP, a template, and a buffer system for the PCR. In the components, the DNA polymerase, the primer, the probe, and the dNTP are all required to be stored at a low temperature. The DNA polymerase is a protein and will loss biological inactivity due to denaturation caused by a high temperature, so the PCR reagent needs to be transported and used at the low temperature.

For RNA detection, RNA is usually reverse transcribed to DNA, and the qPCR is then performed, so reverse transcriptase is required. The reverse transcriptase also needs to be stored at the low temperature, and transportation conditions for the reverse transcriptase are consistent with transportation conditions for DNA polymerase.

A qPCR assay reagent is concise and convenient for pathogen detection and is particularly important for detection of various infectious pathogens, and a quality will remain consistent. However, for an application of a kit in remote areas, economic costs increase due to a need for low temperature transportation, and transport time significantly increases.

### BRIEF SUMMARY OF THE DISCLOSURE

The present disclosure provides a prepackaged polymerase chain reaction (PCR) reagent configured to be transported and stored at room temperature to solve the aforementioned problems.

A technical solution of the present disclosure is as follows:

A prepackaged polymerase chain reaction (PCR) reagent for nucleic acid amplification comprises a PCR reagent, the PCR reagent comprises an antifreeze protein with a concentration of 0.005-0.300 mg/L and an antifreeze protein antibody with a concentration of 0.005-0.300 mg/L, and the PCR reagent is a lyophilized powder.

In the present disclosure, the PCR reagent comprises a conventional PCR reagent, a quantitative real-time PCR reagent (e.g., a fluorescent PCR reagent), etc., but the PCR reagent is not limited thereto. The PCR reagent usually comprises dNTP, an amplification buffer solution, polymerase, Mg′, etc.

In a preferred embodiment, the concentration of the antifreeze protein is 0.01-0.20 mg/L and the concentration of the antifreeze protein antibody is 0.01-0.20 mg/L.

In a preferred embodiment, the concentration of the antifreeze protein is a 0.01-0.15 mg/L and the concentration of the antifreeze protein antibody is 0.01-0.15 mg/L.

In a preferred embodiment, the concentration of the antifreeze protein is 0.01-0.10 mg/L and the concentration of the antifreeze protein antibody is 0.01-0.10 mg/L.

The antifreeze protein (AFP) is a protein or a glycoprotein that inhibits a growth of ice crystals. Studies have found that the AFP is activated to participate to prevent lattice formation due to crystallization caused by a low temperature. Once a temperature rises or pH decreases, the AFP is completely inactivated. In the present disclosure, the antifreeze protein antibody is further added to reduce an infection on PCR from the AFP. An addition of the antifreeze protein offers a possibility to use the prepackaged PCR reagent in various environments (e.g. remote mountainous areas, polar regions, etc.). In the present disclosure, the antifreeze protein comprises at least one of an antifreeze glycoprotein, a type I antifreeze protein, a type II antifreeze protein, a type III antifreeze protein, or a type IV antifreeze protein but is not limited thereto.

In a preferred embodiment, the prepackaged PCR reagent comprises a separately packaged resoluble solution for dissolving the lyophilized powder into a PCR solution. Preferably, the separately packaged resoluble solution is ultrapure water treated using diethyl pyrocarbonate (DEPC).

Another objective of the present disclosure is to provide a method for transporting or preserving the prepackaged PCR reagent for nucleic acid amplification, and the transporting or the preserving is performed at room temperature. Room temperature in the present disclosure is 20-30° C.

Another objective of the present disclosure is to provide a method for preparing a prepackaged polymerase chain reaction (PCR) reagent for nucleic acid amplification, it comprises:

Mixing a PCR reagent comprising an antifreeze protein with a concentration of mg/L and an antifreeze protein antibody with a concentration of 0.005-mg/L to obtain a mixture, placing the mixture in a lyophilizer, pre-freezing at −40° C. to −30° C. for 3-5 hours, then reducing an air pressure of the lyophilizer below 10 Pa, processing in vacuum at −40° C. to −30° C. for 2-3 hours, heating an internal temperature of the lyophilizer in vacuum to 8-12° C., processing in vacuum for 2-3 hours, heating the internal temperature of the lyophilizer to 25-35° C., and processing in vacuum for 2-3 hours.

Compared to the traditional constant temperature lyophilization method, the vacuum treatment using stage heating of the present disclosure can significantly speed up a lyophilization process and improve dryness degree of samples. In a last stage of the stage heating, the mixture is dried at 20-35° C., a temperature of the samples is higher than room temperature (e.g., 20-30° C.), so that some of the samples in which lids cannot be pressed down in the lyophilizer will not be affected by environmental humidity during a process in which the lids are pressed down after the samples are taken out after the lyophilization process, a storage life of the lyophilized powder is prolonged, and a stability of the lyophilization process is ensured.

In a preferred embodiment, dividing the mixture into PCR amplification tubes according to 20-50 μL/tube before the placing the mixture in the lyophilizer. Another objective of the present disclosure is to provide a method for using the prepackaged PCR reagent for nucleic acid amplification, it comprises, dissolving the lyophilized powder with a resoluble solution.

In a preferred embodiment, the resoluble solution is ultrapure water treated using diethyl pyrocarbonate (DEPC).

The present disclosure has the following advantages.

The present disclosure provides a prepackaged PCR reagent for nucleic acid amplification. Steps for use are reduced, a stability of the prepackaged PCR reagent is improved, and storage and transportation at room temperature are achieved. The present disclosure provides a lyophilized and prepackaged reagent for PCR amplification, enabling a nucleic acid amplification reagent to be transported at room temperature after lyophilization. The present disclosure is suitable for a pre-treatment of any fluorescent PCR amplification reagent, and test effects are good. When the prepackaged PCR reagent for nucleic acid amplification is used, only a buffer solution needs to be added for redissolution to obtain a solution, and the solution is then added to a template to be detected for assay with no loss or little loss of sensitivity. A component of a protective agent is simple and low cost.

### BRIEF DESCRIPTION OF THE DRAWING

FIG. 1 illustrates amplification effects of ORF1ab gene by using a standard sample with a concentration of 2×10⁴ CFU/mL in Embodiment 1 of the present disclosure;

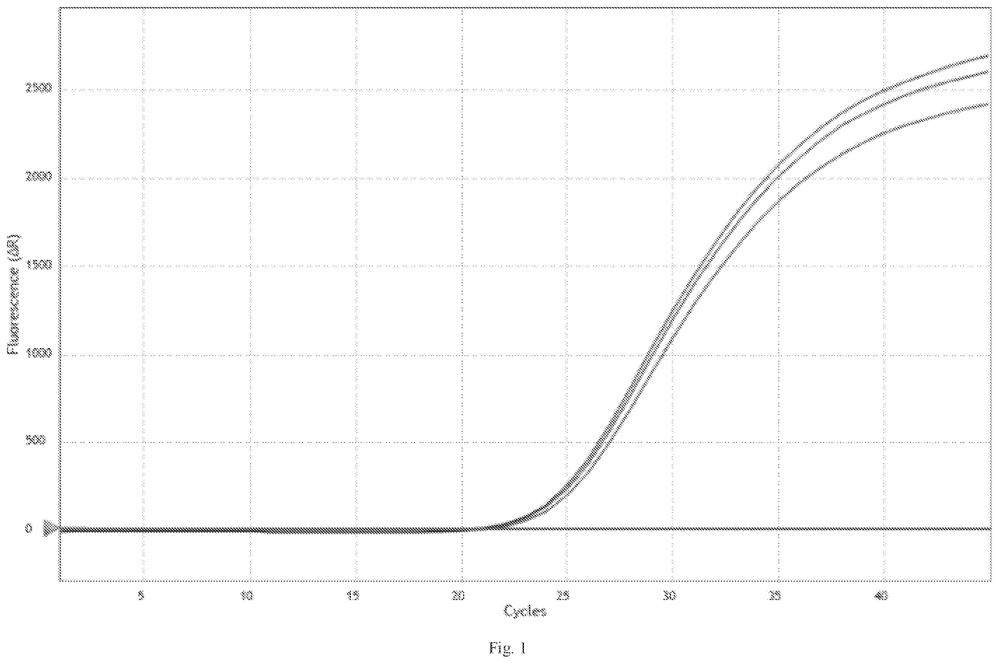

FIG. 2 illustrates amplification effects of N gene by using the standard sample with a concentration of 2×10⁴ CFU/mL in Embodiment 1 of the present disclosure;

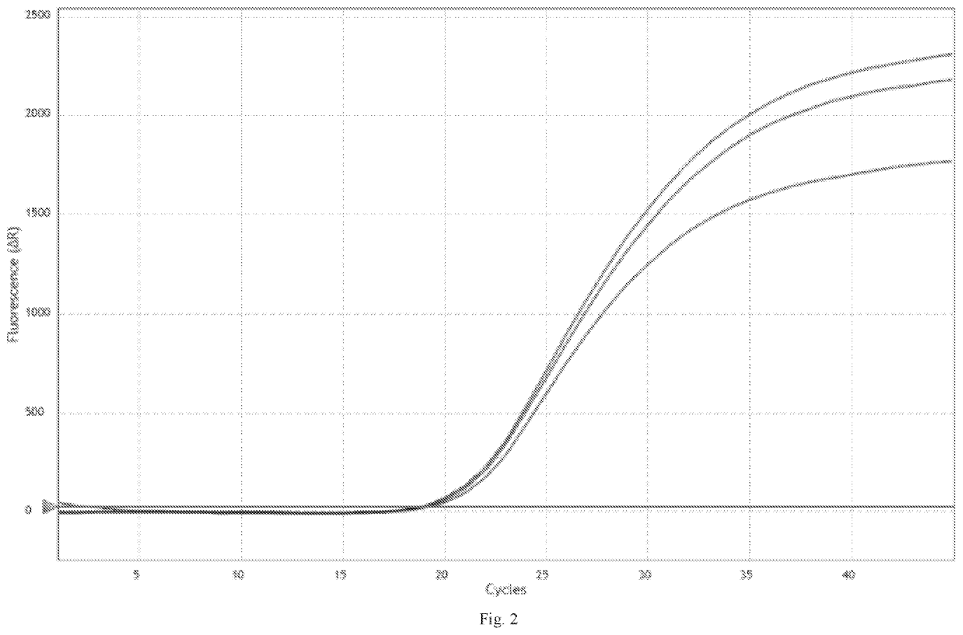

FIG. 3 illustrates amplification effects of the ORF1 ab gene by using a standard sample with a concentration of 2×10³ CFU/mL in Embodiment 1 of the present disclosure; and

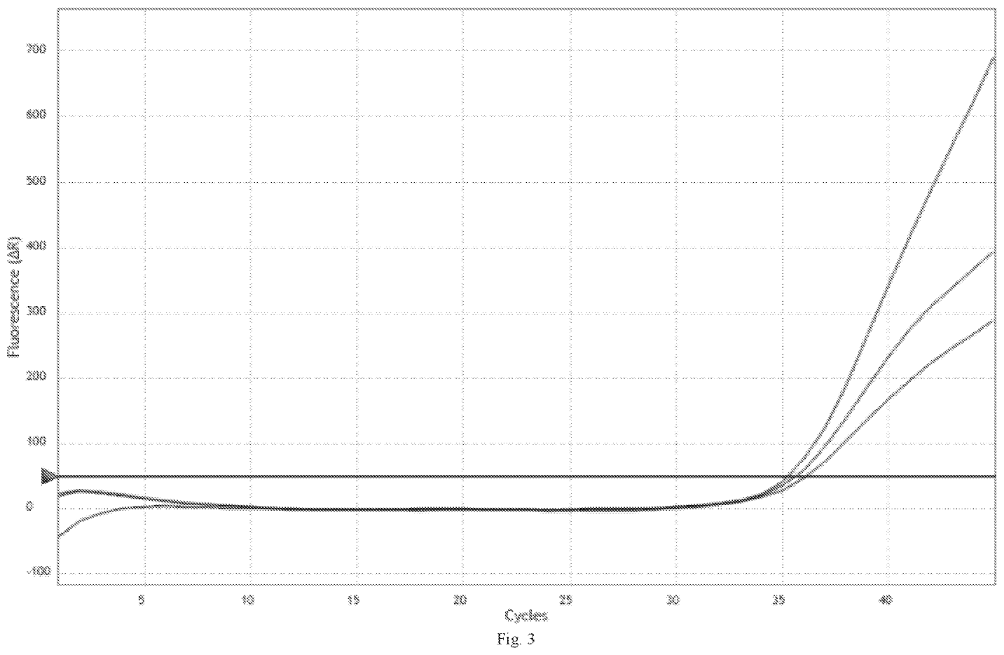

FIG. 4 illustrates amplification effects of the N gene by using the standard sample with a concentration of 2×10³ CFU/mL in Embodiment 1 of the present disclosure.

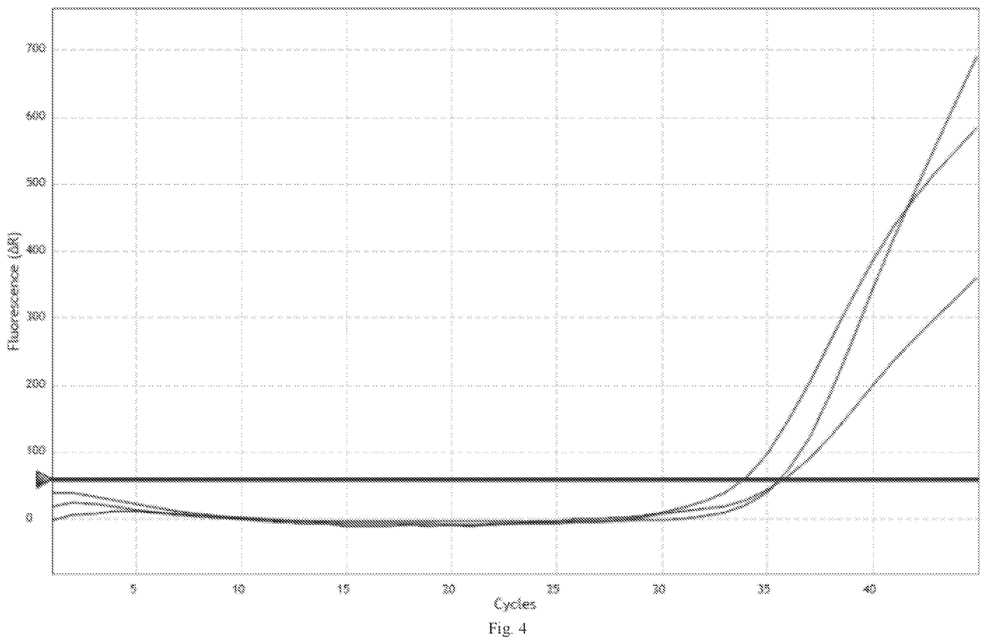

### DETAILED DESCRIPTION OF THE EMBODIMENTS

### Embodiment 1

Coronaviruses (CoVes) are positive-strand RNA viruses with a diameter of 80-120 nm and are important pathogens in humans and many animals to cause a wide variety of acute diseases and chronic diseases. Species of the CoVes are various, of which a genus (including CoV-229E and CoV-NL63) and 13 genus (including CoV-CoV-HKU1, SARS-CoV, MERSCoV, and SARS-CoV-2) infect mammals. SARS-CoV-2 is a currently identified seventh coronavirus infectious to humans with a genome size of 29,891 nucleotides and comprises two untranslated regions (UTR) and a complete open reading frame (ORF) gene for translating 960 amino acids.

The current fluorescent polymerase chain reaction (PCR) test product for SARS-CoV-2 is still a conventional form with 1 tube of buffer solution for liquid reaction and 1 tube of liquid enzyme. Steps for use are cumbersome, storage and transportation are performed at a low temperature, and costs for the storage and transportation are high. The present disclosure discloses a method for lyophilizating a fluorescent PCR reagent to well solve the foregoing problems.

To verify effectiveness of the lyophilization method, the following method is used for preparing and lyophilizating nucleic acid test reagents (which are used in a fluorescent PCR method) of the SARS-CoV-2. The following examples are only used for verification, and the scope of the present disclosure is not limited thereof.

1. Design of Primers and Probes

In the current nucleic acid test of the SARS-CoV-2, an N gene and an ORF1ab gene of the SARS-CoV-2 are mainly detected, so primers and probes for the N gene and the ORF1ab gene are also designed in a nucleic acid test of this example. In addition, an internal reference is required for the nucleic acid test, and RNAse P is used as the internal reference to design the primers and probes in the present disclosure.

Specific sequences are as follows:

An upstream primer of the ORF1ab gene is as follows:

| (SEQ ID NO: 1) | |
|---|---|
| | CCCTGTGGGTTTTACACTTAA |

A downstream primer of the ORF1ab gene is as follows:

| (SEQ ID NO: 2) | |
|---|---|
| | ACGATTGTGCATCAGCTGA |

A probe of the ORF1ab gene is as follows:

(SEQ ID NO: 3)
5′-fluorescent reporter gene-CCGTCTGCGGTATGTGGAAAG
GTTATGG-fluorescent quenching gene-3′

An upstream primer of the N gene is as follows:

| (SEQ ID NO: 4) | |
|---|---|
| | GGGGAACTTCTCCTGCTAGAAT |

A downstream primer of the N gene is as follows:

| (SEQ ID NO: 5) | |
|---|---|
| | CAGACATTTTGCTCTCAAGCTG |

A probe of the N gene is as follows:

(SEQ ID NO: 6)
5′-fluorescent reporter gene-TTGCTGCTGCTTGACAGATT-
fluorescent quenching gene-3′

An upstream primer of the RNAse P is as follows:

| (SEQ ID NO: 7) | |
|---|---|
| | AGATTTGGACCTGCGAGCG |

A downstream primer of the RNAse P is as follows:

| (SEQ ID NO: 8) | |
|---|---|
| | GAGCGGCTGTCTCCACAAGT |

A probe of the RNAse P is as follows:

(SEQ ID NO: 9)
5′-fluorescent reporter gene-TTCTGACCTGAAGGCTCTGCG
CG-fluorescent quenching gene-3′

2. Components of a Fluorescent PCR Reagent are Mixed with the Following Ratios to Obtain a Mixed Solution

A composition is as follows:

| The components of the fluorescent PCR | | |
|---|---|---|
| reagent | Final concentration | |
| 10 × Reaction Mix | 1 X | |
| BSA | 0.3 | mg/mL |
| Formamide | 3% | |
| DTT | 10 | mM |
| dATP | 150 | μM |
| dTTP | 150 | μM |
| dCTP | 150 | μM |
| dGTP | 150 | μM |
| dUTP | 150 | μM |
| HotStart Hi Taq DNA polymerase | 0.025 | U |
| Super-M-MLV reverse transcriptase | 200 | U |
| RNA enzyme inhibitor | 100 | U |
| Primer | 200 | nM |
| Probe | 100 | nM |
| antifreeze protein | 0.05 | mg/L |
| antifreeze protein antibody | 0.05 | mg/L |

The Super-M-MLV reverse transcriptase is purchased from Promega. The antifreeze protein is a type IV antifreeze protein, a gene sequence of the type IV antifreeze protein is shown in registration No. Q8JI37 (Genebank), Lee and Kim (Cloning, expression, and activity of the type IV antifreeze protein from cultured subtropical olive flounder (Paralichthys olivaceus)[J]. Fisheries and Aquatic Sciences (2016) 19:33) disclose a method for preparing the type IV antifreeze protein. The antifreeze protein antibody is purchased from Antibodies-online.com, and the rest of the fluorescent PCR reagent is purchased from Thermo. The mixed solution is divided into 0.2 mL PCR amplification tubes or 8-tube strips with 50 μL/tube to obtain divided fluorescent PCR reagents.

The divided fluorescent PCR reagents are pre-frozen in a lyophilizer at −35° C. for 4 hours. An air pressure in the lyophilizer is reduced to below 10 Pa, and the divided fluorescent PCR reagents are processed in vacuum at −35° C. for 2-3 hours. An internal temperature of the lyophilizer is heated to 10° C. in vacuum, and the divided fluorescent PCR reagents are processed for 2 hours. The internal temperature of the lyophilizer is heated to 30° C. in vacuum, and the divided fluorescent PCR reagents are processed for 3 hours to obtain prepared lyophilized powder of the fluorescent PCR reagent.

Compared to the traditional constant temperature lyophilization method, the vacuum treatment using stage heating of the present disclosure can significantly speed up a lyophilization process and improve dryness degree of samples. In a last stage of the stage heating, the divided fluorescent PCR reagents are dried at 30° C., enabling a temperature of the samples to be higher than room temperature (e.g., higher than 20-30° C.), so that some of the samples in which lids cannot be pressed down in the lyophilizer will not be affected by environmental humidity during a process in which the lids are pressed down after the samples are taken out after the lyophilization process. A storage life of the lyophilized powder is prolonged, and a stability of the lyophilization process is ensured.

After the lyophilization process is complete, lids of the PCR amplification tubes or the 8-tube strips are pressed down, and the prepared lyophilized powder of the fluorescent PCR reagent is stored at room temperature (e.g., 20-30° C.) using a light blocking structure.

The lyophilized powder of the fluorescent PCR reagent is dissolved in ultrapure water treated using diethyl pyrocarbonate (DEPC) and then assayed.

The lyophilized powder of the fluorescent PCR reagent is verified using different concentrations of positive quality controls, and a group of liquid products of the fluorescent PCR reagent is prepared to be used as a control reagent.

Conditions of the amplification reaction are as follows:

| | Number of | Temperature | | Collected |
|---|---|---|---|---|
| Steps | cycles | (° C.) | Time (min:sec) | fluorescent signals |
| 1 | 1 | 48 | 15:00 | no |
| | | 94 | 2:00 | no |
| 2 | 45 | 94 | 0:15 | no |
| | | 58 | 0:40 | yes |

Analysis of Results

Cycle threshold (Ct) values in the amplification reaction are as follows:

Ct values

| Samples | genes | 1 | 2 | 3 |
|---|---|---|---|---|
| 2 × 10⁴ CFU/mL | ORF1ab | 21.62 | 20.91 | 20.01 |
| | N | 19.31 | 19.05 | 18.85 |
| 2 × 10³ CFU/mL | ORF1ab | 35.72 | 35.34 | 36.20 |
| | N | 35.78 | 35.68 | 33.87 |

### Embodiment 2

The lyophilized powder of the fluorescent PCR reagent in Embodiment 1 is used as a control group, and unlyophilized product of the fluorescent PCR reagent is used as an experimental group. The control group and the experimental group are stored at room temperature (e.g., 20-30° C.) for 48 hours for a quantitative polymerase-chain-reaction (qPCR) assay, and a test method of the qPCR assay is according to Embodiment 1.

Results

| Groups | Genes | Ct values | | | Average Ct value |
|---|---|---|---|---|---|
| Control | ORF1ab | 21.87 | 20.98 | 21.45 | 21.43 |
| group | N | 19.76 | 19.09 | 20.32 | 19.72 |
| Experimental | ORF1ab | 37.21 | 37.90 | — | 37.56 |
| group | N | 38.34 | — | 39.98 | 39.16 |

### Embodiment 3

The lyophilized powder of the fluorescent PCR reagent in Embodiment 1 is placed at 70° C., 0° C., and −20° C. to respectively simulate stability at a high temperature, a low temperature, and a very low temperature. The lyophilized powder is respectively placed at 70° C., 0° C., and −20° C. for 3 hours for a quantitative polymerase-chain-reaction (qPCR) assay, and a test method of the qPCR assay is according to Embodiment 1.

Results

| Temperature | Genes | Ct values | | | Average Ct value |
|---|---|---|---|---|---|
| 70° C. | ORF1ab | 37.21 | 37.90 | — | 37.56 |
| | N | 38.34 | — | 39.98 | 39.16 |
| 0 | ORF1ab | 22.32 | 21.56 | 21.98 | 21.95 |
| | N | 19.65 | 19.32 | 19.90 | 19.62 |
| −20° C. | ORF1ab | 21.89 | 20.12 | 21.98 | 21.33 |
| | N | 20.89 | 21.01 | 20.56 | 20.82 |

### Embodiment 4

The fluorescent PCR reagent with and without the antifreeze protein are simultaneously lyophilized and are respectively used as a control group and an experimental group for a quantitative polymerase-chain-reaction (qPCR) assay, and a test method of the qPCR assay is according to Embodiment 1.

Results

| Groups | Genes | Ct values | | | Average Ct value |
|---|---|---|---|---|---|
| Control | ORF1ab | 21.59 | 22.87 | 20.45 | 21.64 |
| group | N | 20.08 | 20.45 | 21.43 | 20.65 |
| Experimental | ORF1ab | 36.89 | 37.98 | 37.21 | 37.36 |
| group | N | 38.09 | 37.28 | 35.67 | 37.01 |

### Embodiment 5

The lyophilized powder of the fluorescent PCR reagent in Embodiment 1 and the existing commercial one-step qPCR test kit (which is purchased from Qiagen) are simultaneously placed at room temperature (e.g., 20-30° C.) for 2 weeks and are used as a control group and an experimental group for a quantitative polymerase-chain-reaction (qPCR) assay, and test methods of the qPCR assay are respectively according to Embodiment 1 and instructions of the test kit.

Results

| Groups | Genes | Ct values | | | Average Ct value |
|---|---|---|---|---|---|
| Control | ORF1ab | 28.56 | 29.14 | 29.56 | 29.08 |
| group | N | 28.12 | 27.34 | 29.56 | 28.34 |
| Experimental | ORF1ab | 37.89 | 38.98 | — | 38.43 |
| group | N | 38.27 | 39.89 | 39.01 | 39.08 |

In summary, in the present disclosure, the antifreeze protein and the antifreeze protein antibody are added into a PCR system, so that nucleic acid amplification reagent (i.e., the fluorescent PCR reagent) can be transported under room temperature conditions after being lyophilized. The present disclosure is suitable for a pretreatment of any fluorescent PCR amplification reagent with good test effects and without interference to test results. Thus, it is intended that the present disclosure cover any modifications and variations provided they are made without departing from the appended claims and the specification of the present disclosure.

What is claimed is:

1. A prepackaged polymerase chain reaction (PCR) reagent for nucleic acid amplification, comprising:
a PCR reagent, wherein:
the PCR reagent comprises an antifreeze protein with a concentration of 0.005-0.300 mg/L and an antifreeze protein antibody with a concentration of 0.005-0.300 mg/L, and
the PCR reagent is a lyophilized powder.

2. The prepackaged PCR reagent for nucleic acid amplification according to claim 1, wherein the concentration of the antifreeze protein is 0.01-0.20 mg/L and the concentration of the antifreeze protein antibody is 0.01-0.20 mg/L.

3. The prepackaged PCR reagent for nucleic acid amplification according to claim 1, wherein the concentration of the antifreeze protein is a 0.01-0.15 mg/L and the concentration of the antifreeze protein antibody is 0.01-0.15 mg/L.

4. The prepackaged PCR reagent for nucleic acid amplification according to claim 1, wherein the concentration of the antifreeze protein is 0.01-0.10 mg/L and the concentration of the antifreeze protein antibody is 0.01-0.10 mg/L.

5. The prepackaged PCR reagent for nucleic acid amplification according to claim 1, comprising:
a separately packaged resoluble solution for dissolving the lyophilized powder into a PCR solution.

6. A method for transporting or preserving the prepackaged PCR reagent for nucleic acid amplification according to claim 1, wherein the transporting or the preserving is performed at room temperature.

7. A method for preparing a prepackaged polymerase chain reaction (PCR) reagent for nucleic acid amplification, comprising:
mixing a PCR reagent comprising an antifreeze protein with a concentration of 0.005-0.300 mg/L and an antifreeze protein antibody with a concentration of 0.005-0.300 mg/L to obtain a mixture,
placing the mixture in a lyophilizer,
pre-freezing at −40° C. to −30° C. for 3-5 hours,
then reducing an air pressure of the lyophilizer below 10 Pa,
processing in vacuum at −40° C. to −30° C. for 2-3 hours,
heating an internal temperature of the lyophilizer in vacuum to 8-12° C.,
processing in vacuum for 2-3 hours,
heating the internal temperature of the lyophilizer to 25-35° C., and
processing in vacuum for 2-3 hours.

8. The method for preparing the prepackaged PCR reagent for nucleic acid amplification according to claim 7, comprising:
dividing the mixture into PCR amplification tubes according to 20-50 μL/tube before the placing the mixture in the lyophilizer.

9. A method for using the prepackaged PCR reagent for nucleic acid amplification according to claim 1, comprising:
dissolving the lyophilized powder with a resoluble solution.

10. The method for using the prepackaged PCR reagent for nucleic acid amplification according to claim 9, wherein the resoluble solution is ultra-pure water treated using diethyl pyrocarbonate (DEPC).

11. A prepackaged polymerase chain reaction (PCR) reagent for nucleic acid amplification, comprising:
a PCR reagent, wherein:
the PCR reagent comprises an antifreeze protein and an antifreeze protein antibody.

12. The prepackaged PCR reagent for nucleic acid amplification according to claim 11, wherein a concentration of the antifreeze protein is 0.005-0.300 mg/L and a concentration of the antifreeze protein antibody is 0.005-0.300 mg/L.

13. The prepackaged PCR reagent for nucleic acid amplification according to claim 11, wherein the PCR reagent is lyophilized powder.

14. The prepackaged PCR reagent for nucleic acid amplification according to claim 11, wherein the antifreeze protein comprises at least one of antifreeze glycoprotein, type I antifreeze protein, type II antifreeze protein, type III antifreeze protein, or type IV antifreeze protein.


SEQUENCE LISTING (9 sequences)

SEQ ID NO: 1 - DNA, 21 bp, synthetic construct
  misc_feature 1..21: note = Synthetic
  source 1..21: mol_type = other DNA
ccctgtgggttttacacttaa

SEQ ID NO: 2 - DNA, 19 bp, synthetic construct
  misc_feature 1..19: note = Synthetic
  source 1..19: mol_type = other DNA
acgattgtgcatcagctga

SEQ ID NO: 3 - DNA, 28 bp, synthetic construct
  misc_feature 1..28: note = Synthetic
  source 1..28: mol_type = other DNA
ccgtctgcggtatgtggaaaggttatgg

SEQ ID NO: 4 - DNA, 22 bp, synthetic construct
  misc_feature 1..22: note = Synthetic
  source 1..22: mol_type = other DNA
ggggaacttctcctgctagaat

SEQ ID NO: 5 - DNA, 22 bp, synthetic construct
  misc_feature 1..22: note = Synthetic
  source 1..22: mol_type = other DNA
cagacattttgctctcaagctg

SEQ ID NO: 6 - DNA, 20 bp, synthetic construct
  misc_feature 1..20: note = Synthetic
  source 1..20: mol_type = other DNA
ttgctgctgcttgacagatt

SEQ ID NO: 7 - DNA, 19 bp, synthetic construct
  misc_feature 1..19: note = Synthetic
  source 1..19: mol_type = other DNA
agatttggacctgcgagcg

SEQ ID NO: 8 - DNA, 20 bp, synthetic construct
  misc_feature 1..20: note = Synthetic
  source 1..20: mol_type = other DNA
gagcggctgtctccacaagt

SEQ ID NO: 9 - DNA, 23 bp, synthetic construct
  misc_feature 1..23: note = Synthetic
  source 1..23: mol_type = other DNA
ttctgacctgaaggctctgcgcg